(19) United States
(12) Patent Application Publication
(10) Pub. No.: US 2010/0160626 A1
(43) Pub. Date: Jun. 24, 2010

(54) Process For The Preparation Of 3,7-Dihydroxy-1,5-Diazacyclooctanes

(75) Inventors: Emma Anderson, Leicestershire (GB); David Cladingboel, Leicestershire (GB); Gareth Ensor, Leicestershire (GB); David Holmes, Middlesbrough (GB); Mark Purdie, Leicestershire (GB)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 11/993,032

(22) PCT Filed: Jun. 12, 2006
(86) PCT No.: PCT/SE06/00689

§ 371 Date: Apr. 17, 2008

(30) Foreign Application Priority Data
Jun. 20, 2005 (SE) 0501431-1
Dec. 15, 2005 (SE) 0502772-7

(51) Int. Cl. C07D 245/02; C07D 498/08; C07D 407/12

(52) U.S. Cl. 540/470; 544/74; 549/513

(57) ABSTRACT

There is provided processes for the preparation of various compounds, including compounds of formulae I, IV and VII,

or salts and/or solvates thereof, which compounds are useful intermediates in the synthesis of certain oxabispidines having antiarrhythmic activity, such as compounds of formula XI wherein R [symbol] 1?, R [symbol] 15?, R [symbol] 16?, R [symbol] 18? and D have meanings given in the description.

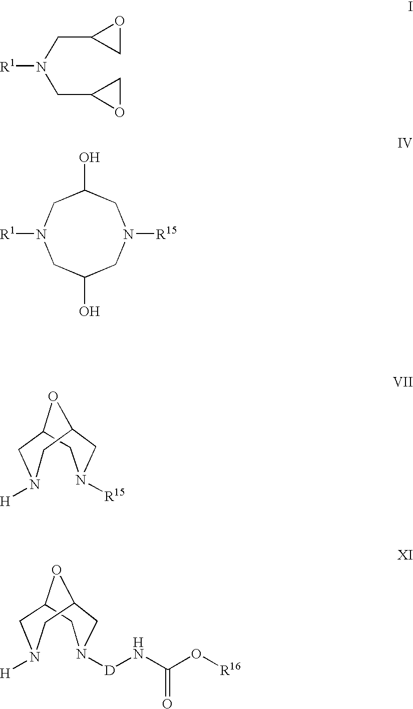

### FIELD OF THE INVENTION

The invention relates to a novel process for the preparation of 3,7-dihydroxy-1,5-diazacyclooctanes, which compounds may be further converted by a novel process to oxabispidines.

### BACKGROUND AND PRIOR ART

The number of documented compounds including the 9-oxa-3,7-diazabicyclo-[3.3.1]nonane (oxabispidine) structure is very few. As a result, there are very few known processes that efficiently provide compounds comprising the oxabispidine ring system wherein one or both of the nitrogen atoms of the oxabispidine are substituted.

Certain oxabispidine compounds are disclosed in Chem. Ber. 96(11), 2827 (1963) as intermediates in the synthesis of 1,3-diaza-6-oxa-adamantanes.

Hemiacetals (and related compounds) having the oxabispidine ring structure are disclosed in J. Org. Chem. 31, 277 (1966), ibid. 61(25), 8897 (1996), ibid. 63(5), 1566 (1998) and ibid. 64(3), 960 (1999) as unexpected products from the oxidation of 1,5-diazacyclooctane-1,3-diols or the reduction of 1,5-diazacyclooctane-1,3-diones.

1,3-Dimethyl-3,7-ditosyl-9-oxa-3,7-diazabicyclo[3.3.1]nonane is disclosed in J. Org. Chem. 32, 2425 (1967) as a product from the attempted acetylation of trans-1,3-dimethyl-1,5-ditosyl-1,5-diazacyclooctane-1,3-diol.

International patent applications WO 01/28992, WO 02/83690, WO 02/28864 and WO 2004/035592 describe the synthesis of a wide range of oxabispidines (or intermediates useful in the production of such compounds), which compounds are indicated as being useful in the treatment of cardiac arrhythmias. Various processes for the preparation of oxabispidines are disclosed in these applications including their preparation from 3,7-dihydroxy-1,5-diazacyclooctanes via a dehydrative cyclisation reaction. The preparation of 3,7-dihydroxy-1,5-diazacyclooctanes from the reaction of an N,N-bis(2-oxiranylmethyl)amine with another amine is also disclosed. The preparation of N,N-bis(2-oxiranylmethyl)-amines from the reaction of an amine with an epihalohydrin is further disclosed.

However, there is no disclosure in any of the above-mentioned applications of the reaction of an amine with an epihalohydrin (or equivalent nucleophile) to produce a N,N-bis(2-oxiranylmethyl)amine in the presence of an aqueous solvent and base, such that the reaction mixture is substantially maintained at a pH of between 10.0 and 13.0 throughout the course of the reaction. Nor is there any suggestion of the reaction between a N,N-bis(2-oxiranylmethyl)amine with an amine to produce a 3,7-dihydroxy-1,5-diazacyclooctane, wherein each of the reagents is added to the reaction vessel separately, simultaneously and at a substantially equivalent rate of moles per minute. Further, there is no disclosure or suggestion in any of the above applications of a sulfuric acid-catalysed deprotection of an N-protected oxabispidine, nor of a “one-pot” dehydrative cyclisation and deprotection of an N-protected 3,7-dihydroxy-1,5-diazacyclooctane, wherein treatment with acid effects the cyclisation with concomitant deprotection of the nitrogen protective group to provide an oxabispidine in which one of the nitrogen atoms is unsubstituted.

We have now found that the above-mentioned compounds, which are useful intermediates in the synthesis of pharmacologically active oxabispidines, may be readily and efficiently prepared via such processes.

### DISCLOSURE OF THE INVENTION

According to a first aspect of the invention, there is provided a process for the preparation of a compound of formula I,

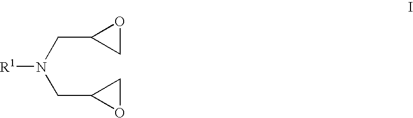

or a salt and/or solvate thereof,
wherein R¹ represents an amino protective group or a structural fragment of formula Ia,

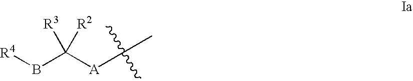

in which
R² represents H, halo, C₁₋₆ alkyl, —OR⁵, -E-N(R⁶)R⁷ or, together with R³, represents ═O;
R³ represents H, C₁₋₆ alkyl or, together with R², represents ═O;
R⁵ represents H, C₁₋₆ alkyl, -E-aryl, -E-Het¹, —C(O)R^{8a}, —C(O)OR^{8b} or —C(O)N(R^{9a})R^{9b};
R⁶ represents H, C₁₋₆ alkyl, -E-aryl, -E-Het¹, —C(O)R^{8a}, —C(O)OR^{8b}, —S(O)₂R^{8c}, —[C(O)]ₚN(R^{9a})R^{9b} or —C(NH)NH₂;
R⁷ represents H, C₁₋₆ alkyl, -E-aryl or —C(O)R^{8d};
R^{8a} to R^{8d} independently represent, at each occurrence when used herein, C₁₋₆ alkyl (optionally substituted by one or more substituents selected from halo, aryl and Het²), aryl, Het³, or R^{8a} and R^{8d} independently represent H;
R^{9a} and R^{9b} independently represent, at each occurrence when used herein, H or C₁₋₆ alkyl (optionally substituted by one or more substituents selected from halo, aryl and Het⁴), aryl, Het⁵, or together represent C₃₋₆ alkylene, optionally interrupted by an O atom;
E represents, at each occurrence when used herein, a direct bond or C₁₋₄ alkylene;
p represents 1 or 2;
A represents a direct bond, -J-, -J-N(R^{10a})—, -J-S(O)₂N(R^{10b})—, -J-N(R^{10c})S(O)₂— or -J-O— (in which latter four groups -J is attached to the oxabispidine nitrogen);
B represents —Z—{[C(O)]ₐC(H)(R^{11a})}_{b}—, —Z—[C(O)]_{c}N(R^{11b})—, —Z—N(R^{11c})S(O)₂—, —Z—S(O)₂N(R^{11d})—, —Z—S(O)ₙ—, —Z—O— (in which latter six groups, Z is attached to the carbon atom bearing R² and R³), —N(R^{11e})—Z—, —N(R^{11f})S(O)₂—Z—, —S(O)₂N(R^{11g})—Z— or —N(R^{11h})C(O)O—Z— (in which latter four groups, Z is attached to the R⁴ group);
J represents C₁₋₆ alkylene optionally interrupted by —S(O)₂N(R^{10d})— or —N(R^{10e})S(O)₂— and/or optionally substituted by one or more substituents selected from —OH, halo and amino;
Z represents a direct bond or C₁₋₄ alkylene, optionally interrupted by —N(R¹¹ⁱ)S(O)₂— or —S(O)₂N(R^{11j})—;
a, b and c independently represent 0 or 1;
n represents 0, 1 or 2;
R^{10a} to R^{10e} independently represent, at each occurrence when used herein, H or C₁₋₆ alkyl;
R^{11a} represents H or, together with a single ortho-substituent on the R⁴ group (ortho-relative to the position at which the B group is attached), R^{11a} represents C₂₋₄ alkylene optionally interrupted or terminated by O, S, N(H) or N(C₁₋₆ alkyl);
R^{11b} represents H, C₁₋₆ alkyl or, together with a single ortho-substituent on the R⁴ group (ortho-relative to the position at which the B group is attached), R^{11b} represents C₂₋₄ alkylene;
R^{11c} to R^{11j} independently represent, at each occurrence when used herein, H or C₁₋₆ alkyl;
R⁴ represents phenyl or pyridyl, both of which groups are optionally substituted by one or more substituents selected from —OH, cyano, halo, nitro, C₁₋₆ alkyl (optionally terminated by —N(H)C(O)OR^{12a}), C₁₋₆ alkoxy, —N(R^{13a})R^{13b}, —C(O)R^{13c}, —C(O)OR^{13d}, —C(O)N(R^{13e})R^{13f}, —N(R^{13g})C(O)R^{13h}, —N(R¹³ⁱ)C(O)N(R^{13j})R^{13k}, —N(R^{13m})S(O)₂R^{12b}, —S(O)₂N(R¹³ⁿ)R^{13o}, —S(O)₂R^{12c}, —OS(O)₂R^{12d} and/or aryl;
and an ortho-substituent (ortho-relative to the attachment of B) may
(i) together with R^{11a}, represent C₂₋₄ alkylene optionally interrupted or terminated by O, S, N(H) or N(C₁₋₄ alkyl), or
(ii) together with R^{11b}, represent C₂₋₄ alkylene;
   R^{12a} to R^{12d} independently represent C₁₋₆ alkyl;
   R^{13a} and R^{13b} independently represent H, C₁₋₆ alkyl or together represent C₃₋₆ alkylene, resulting in a four- to seven-membered nitrogen-containing ring;
   R^{13c} to R^{13o} independently represent H or C₁₋₆ alkyl; and
   Het¹ to Het⁵ independently represent, at each occurrence when used herein, five- to twelve-membered heterocyclic groups containing one or more heteroatoms selected from oxygen, nitrogen and/or sulfur, which heterocyclic groups are optionally substituted by one or more substituents selected from ═O, —OH, cyano, halo, nitro, C₁₋₆ alkyl, C₁₋₆ alkoxy, aryl, aryloxy, —N(R^{14a})R^{14b}, —C(O)R^{14c}, —C(O)OR^{14d}, —C(O)N(R^{14e})R^{14f}, —N(R^{14g})C(O)R^{14h}, —S(O)₂N(R¹⁴ⁱ)R^{14j}) and/or —N(R^{14k})S(O)₂R^{14l};
   R^{14a} to R^{14l} independently represent C₁₋₆ alkyl, aryl or R^{14a} to R^{14k} independently represent H;
   provided that:
(a) when R³ represents H or C₁₋₆ alkyl; and

A represents -J-N(R^{10a}) or -J-O—, then:
(i) J does not represent C₁ alkylene or 1,1-C₂₋₆ alkylene; and
B does not represent —N(R^{11b})—, —N(R^{11c})S(O)₂—, —S(O)ₙ—, —O—, —N(R^{11e})—Z—, —N(R^{11f})S(O)₂—Z— or —N(R^{11h})C(O)O—Z—; and
(b) when R² represents —OR⁵ or -E-N(R⁶)R⁷ in which E represents a direct bond, then:
   (i) A does not represent a direct bond, -J-N(R^{10a})—, -J-S(O)₂N(R^{12b})— or -J-O—; and
   (ii) B does not represent —N(R^{11b})—, —N(R^{11c})S(O)₂—, —S(O)ₙ—, —O—, —N(R^{11e})—Z, —N(R^{11f})S(O)₂—Z— or —N(R^{11h})C(O)O—Z—; and
(c) when A represents -J-N(R^{10c})S(O)₂, then J does not represent C₁ alkylene or 1,1-C₂₋₆ alkylene; and
(d) when R³ represents H or C₁₋₆ alkyl and A represents -J-S(O)₂N(R^{10b}), then B does not represent —N(R^{11b})—, —N(R^{11c})S(O)₂—, —S(O)ₙ—, —O—, —N(R^{11e})—Z—, —N(R^{11f})S(O)₂—Z— or —N(R^{11h})C(O)O—Z—; and
   wherein each aryl and aryloxy group, unless otherwise specified, is optionally substituted;
   which process comprises reaction in the presence of an aqueous solvent system of: one equivalent of a compound of formula II,

H₂N—R¹ II

or a salt and/or solvate thereof, wherein R¹ is as hereinbefore defined,
with at least two equivalents of a compound of formula III,

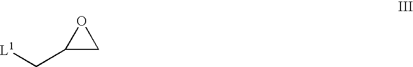

wherein L¹ represents a leaving group,
and at least two equivalents of base,
wherein the reaction is performed by addition of base to an aqueous mixture of the compounds of formulae II and III, the period of base addition comprising:
(a) a first period, during which the pH of the reaction mixture is raised to between pH 10 and pH 13; and then
(b) a second period, during which the pH of the reaction mixture is controlled such that it is maintained between pH 10 and pH 13,
   wherein the time ratio of the first to second period is 1:5 or less,
   which process is hereinafter referred to as “the process of the invention”.

Unless otherwise specified, alkyl groups and alkoxy groups as defined herein may be straight-chain or, when there is a sufficient number (i.e. a minimum of three) of carbon atoms be branched-chain, and/or cyclic. Further, when there is a sufficient number (i.e. a minimum of four) of carbon atoms; such alkyl and alkoxy groups may also be part cyclic/acyclic. Such alkyl and alkoxy groups may also be saturated or, when there is a sufficient number (i.e. a minimum of two) of carbon atoms, be unsaturated and/or interrupted by one or more oxygen and/or sulfur atoms. Unless otherwise specified, alkyl and alkoxy groups may also be substituted by one or more halo, and especially fluoro, atoms.

Unless otherwise specified, alkylene groups as defined herein may be straight-chain or, when there is a sufficient number (i.e. a minimum of two) of carbon atoms, be branched-chain. Such alkylene chains may also be saturated or, when there is a sufficient number (i.e. a minimum of two) of carbon atoms, be unsaturated and/or interrupted by one or more oxygen and/or sulfur atoms. Unless otherwise specified, alkylene groups may also be substituted by one or more halo atoms.

The term “aryl”, when used herein, includes C₆₋₁₃ aryl (e.g. C₆₋₁₀) groups. Such groups may be monocyclic, bicyclic or tricylic and, when polycyclic, be either wholly or partly aromatic. In this respect, C₆₋₁₃ aryl groups that may be mentioned include phenyl, naphthyl, 1,2,3,4-tetrahydronaphthyl, indanyl, indenyl, fluorenyl and the like. For the avoidance of doubt, the point of attachment of substituents on aryl groups may be via any carbon atom of the ring system.

The term “aryloxy”, when used herein includes C₆₋₁₃ aryloxy groups such as phenoxy, naphthoxy, fluorenoxy and the like. For the avoidance of doubt, aryloxy groups referred to herein are attached to the rest of the molecule via the O-atom of the oxy-group.

Unless otherwise specified, aryl and aryloxy groups may be substituted by one or more substituents selected from —OH, cyano, halo, nitro, C₁₋₆ alkyl, C₁₋₆ alkoxy, —N(R^{13a})R^{13b}, —C(O)R^{13c}, —C(O)OR^{13d}, —C(O)N(R^{13e})R^{13f}, —N(R^{13g})C(O)R^{13h}, —N(R^{13m})S(O)₂R^{12b}, —S(O)₂N(R¹³ⁿ)(R^{13o}), —S(O)₂R^{12c} and/or —OS(O)₂R^{12d}, (wherein R^{12b} to R^{12d} and R^{13a} to R^{13o} are as hereinbefore defined). When substituted, aryl and aryloxy groups are preferably substituted by between one and three substituents. For the avoidance of doubt, the point of attachment of substituents on aryl groups may be via any carbon atom of the ring system.

The term “halo”, when used herein, includes fluoro, chloro, bromo and iodo.

Het (Het¹, Het², Het³, Het⁴ and Het⁵) groups that may be mentioned include those containing 1 to 4 heteroatoms (selected from the group oxygen, nitrogen and/or sulfur) and in which the total number of atoms in the ring system are between five and twelve. Het (Het¹, Het², Het³, Het⁴ and Het⁵) groups may be fully saturated, wholly aromatic, partly aromatic and/or bicyclic in character. Heterocyclic groups that may be mentioned include 1-azabicyclo[2.2.2]octanyl, benzimidazolyl, benzisoxazolyl, benzodioxanyl, benzodioxepanyl, benzodioxolyl, benzofuranyl, benzofurazanyl, benzomorpholinyl, 2,1,3-benzoxadiazolyl, benzoxazinonyl, benzoxazol-idinyl, benzoxazolyl, benzopyrazolyl, benzo[e]pyrimidine, 2,1,3-benzothiadiazolyl, benzothiazolyl, benzothienyl, benzotriazolyl, chromanyl, chromenyl, cinnolinyl, 2,3-dihydrobenzimidazolyl, 2,3-dihydrobenzo[b]furanyl, 1,3-dihydrobenzo[c]furanyl, 2,3-dihydropyrrolo[2,3-b]pyridyl, dioxanyl, furanyl, hexahydropyrimidinyl, hydantoinyl, imidazolyl, imidazo[1,2-a]pyridyl, imidazo-[2,3-b]thiazolyl, indolyl, isoquinolinyl, isoxazolyl, maleimido, morpholinyl, oxadiazolyl, 1,3-oxazinanyl, oxazolyl, phthalazinyl, piperazinyl, piperidinyl, purinyl, pyranyl, pyrazinyl, pyrazolyl, pyridyl, pyrimidinyl, pyrrolidinonyl, pyrrolidinyl, pyrrolinyl, pyrrolo[2,3-b]pyridyl, pyrrolo[5,1-b]pyridyl, pyrrolo[2,3-c]pyridyl, pyrrolyl, quinazolinyl, quinolinyl, sulfolanyl, 3-sulfolenyl, 4,5,6,7-tetrahydrobenzimidazolyl, 4,5,6,7-tetrahydrobenzopyrazolyl, 5,6,7,8-tetrahydrobenzo[e]pyrimidine, tetrahydrofuranyl, tetrahydropyranyl, 3,4,5,6-tetrahydropyridyl, 1,2,3,4-tetrahydropyrimidinyl, 3,4,5,6-tetrahydropyrimidinyl, thiadiazolyl, thiazolidinyl, thiazolyl, thienyl, thieno[5,1-c]pyridyl, thiochromanyl, triazolyl, 1,3,4-triazolo[2,3-b]pyrimidinyl and the like.

Substituents on Het (Het¹, Het², Het³, Het⁴ and Het⁵) groups may, where appropriate, be located on any atom in the ring system including a heteroatom. The point of attachment of Het (Het¹, Het², Het³, Het⁴ and Het⁵) groups may be via any atom in the ring system including (where appropriate) a heteroatom, or an atom on any fused carbocyclic ring that may be present as part of the ring system. Het (Het¹, Het², Het³, Het⁴ and Het⁵) groups may also be in the N- or S-oxidised form.

Salts of the compounds of formulae I and II that may be mentioned include acid addition salts. Solvates that may be mentioned include hydrates.

Compounds employed in or produced by the processes described herein (i.e. those involving the process of the invention) may exhibit tautomerism. The process of the invention therefore encompasses the use or production of such compounds in any of their tautomeric forms, or in mixtures of any such forms.

Similarly, the compounds employed in or produced by the processes described herein (i.e. those involving the process of the invention) may also contain one or more asymmetric carbon atoms and may therefore exist as enantiomers or diastereoisomers, and may exhibit optical activity. The process of the invention thus encompasses the use or production of such compounds in any of their optical or diastereoisomeric forms, or in mixtures of any such forms.

Abbreviations are listed at the end of this specification.

As used herein, the term “amino protective group” includes groups mentioned in “Protective Groups in Organic Synthesis”, 3^{rd} edition, T. W. Greene & P. G. M. Wutz, Wiley-Interscience (1999), in particular those mentioned in the chapter entitled “Protection for the Amino Group” (see pages 494 to 502) of that reference, the disclosure in which document is hereby incorporated by reference. Specific examples of amino protective groups thus include:
(a) those which form carbamate groups (e.g. to provide methyl, cyclopropylmethyl, 1-methyl-1-cyclopropylmethyl, diisopropyl-methyl, 9-fluorenylmethyl, 9-(2-sulfo)fluorenylmethyl, 2-furanylmethyl, 2,2,2-trichloroethyl, 2-haloethyl, 2-trimethylsilylethyl, 2-methylthioethyl, 2-methylsulfonylethyl, 2(p-toluenesulfonyl)ethyl, 2-phosphonioethyl, 1,1-dimethylpropynyl, 1,1-dimethyl-3-(N,N-dimethylcarboxamido)propyl, 1,1-dimethyl-3-(N,N-diethylamino)propyl, 1-methyl-1-(1-adamantyl)ethyl, 1-methyl-1-phenyl ethyl, 1-methyl-1-(3,5-dimethoxyphenyl)ethyl, 1-methyl-1-(4-biphenylyl)ethyl, 1-methyl-1-(p-phenylazophenyl)ethyl, 1,1-dimethyl-2-haloethyl, 1,1-dimethyl-2,2,2-trichloroethyl, 1,1-dimethyl-2-cyanoethyl, isobutyl, t-butyl, t-amyl, cyclobutyl, 1-methylcyclobutyl, cyclopentyl, cyclohexyl, 1-methylcyclohexyl, 1-adamantyl, isobornyl, vinyl, allyl, cinnamyl, phenyl, 2,4,6-tri-t-butylphenyl, m-nitrophenyl, S-phenyl, 8-quinolinyl, N-hydroxypiperidinyl, 4-(1,4-dimethylpiperidinyl), 4,5-diphenyl-3-oxazolin-2-one, benzyl, 2,4,6-trimethylbenzyl, p-methoxybenzyl, 3,5-dimethoxybenzyl, p-decyloxybenzyl, p-nitrobenzyl, o-nitrobenzyl, 3,4-dimethoxy-6-nitrobenzyl, p-bromobenzyl, chlorobenzyl, 2,4-dichlorobenzyl, p-cyanobenzyl, o-(N,N-dimethylcarboxamidobenzyl)-benzyl, m-chloro-p-acyloxybenzyl, p-(dihydroxyboryl)benzyl, p-(phenylazo)benzyl, p-(p′-methoxyphenylazo)benzyl, 5-benzisoxazolylmethyl, 9-anthrylmethyl, diphenylmethyl, phenyl(o-nitrophenyl)methyl, di(2-pyridyl)methyl, 1-methyl-1-(4-pyridyl)-ethyl, isonicotinyl, or S-benzyl, carbamate groups);
(b) those which form amide groups (e.g. to provide N-formyl, N-acetyl, N-chloroacetyl, N-dichloro-acetyl, N-trichloroacetyl, N-trifluoroacetyl, N-o-nitrophenylacetyl, N-o-nitrophenoxyacetyl, N-acetoacetyl, N-acetylpyridinium, N-3-phenylpropionyl, N-3-(p-hydroxyphenyl)propionyl, N-3-(o-nitrophenyl)propionyl, N-2-methyl-2-(o-nitrophenoxy)propionyl, N-2-methyl-2-(o-phenylazophenoxy)propionyl, N-4-chlorobutyryl, N-isobutyryl, N-o-nitrocinnamoyl, N-picolinoyl, N—(N′-acetylmethionyl), N—(N′-benzoylphenylalanyl), N-benzoyl, N-p-phenylbenzoyl, N-p-methoxybenzoyl, N-o-nitrobenzoyl, or N-o-(benzoyloxymethyl)benzoyl, amide groups);
(c) those which form N-alkyl groups (e.g. N-allyl, N-phenacyl, N-3-acetoxypropyl, N-(4-nitro-1-cyclohexyl-2-oxo-pyrrolin-3-yl), N-methoxymethyl, N-chloroethoxymethyl, N-benzyloxymethyl, N-pivaloyloxymethyl, N-2-tetrahydropyranyl, N-2,4-dinitrophenyl, N-benzyl, N-3,4-di-methoxybenzyl, N-o-nitrobenzyl, N-di(p-methoxyphenyl)methyl, N-triphenylmethyl, N-(p-methoxyphenyl)-diphenylmethyl, N-diphenyl-4-pyridylmethyl, N-2-picolyl N′-oxide, or N-dibenzosuberyl, groups);
(d) those which form N-phosphinyl and N-phosphoryl groups (e.g. N-diphenylphosphinyl, N-dimethylthiophosphinyl, N-diphenylthiophosphinyl, N-diethylphosphoryl, N-dibenzylphosphoryl, or N-phenylphosphoryl, groups);
(e) those which form N-sulfenyl groups (e.g. N-benzenesulfenyl, N-o-nitrobenzenesulfenyl, N-2,4-dinitrobenzenesulfenyl, N-pentachlorobenzenesulfenyl, N-2-nitro-4-methoxybenzenesulfenyl, or N-triphenylmethylsulfenyl, groups);
(f) those which form N-sulfonyl groups (e.g. N-benzenesulfonyl, N-p-nitrobenzenesulfonyl, N-p-methoxybenzenesulfonyl, N-2,4,6-trimethylbenzenesulfonyl, N-toluenesulfonyl, N-benzylsulfonyl, N-p-methylbenzylsulfonyl, N-trifluoromethylsulfonyl, or N-phenacylsulfonyl, groups); and
(g) that which forms the N-trimethylsilyl group.

Preferred values of compounds of formula I include those in which R¹ represents an amino protective group or a structural fragment of formula Ia in which:

R² represents H, halo, C₁₋₃ alkyl, —OR⁵, —N(H)R⁶ or, together with R³, represents ═O;
R³ represent H, C₁₋₃ alkyl or, together with R², represents ═O;
R⁵ represents H, C₁₋₆ alkyl, -E-(optionally substituted phenyl) or -E-Het¹;
R⁶ represents H, C₁₋₆ alkyl, -E-(optionally substituted phenyl), —C(O)R^{8a}, —C(O)OR^{8b}, S(O)₂R^{8c}, —C(O)N(R^{9a})R^{9b} or —C(NH)NH₂;
R^{8a} to R^{8c} independently represent C₁₋₆ alkyl, or R^{8a} represents H;
R^{9a} and R^{9b} independently represent H or C₁₋₄ alkyl;
E represents, at each occurrence when used herein, a direct bond or C₁₋₂ alkylene;
A represents -G-, -J-N(R¹⁰)— or -J-O—;
B represents —Z—, —Z—N(R¹¹)—, —Z—S(O)ₙ—, —Z—O—;
G represents C₁₋₄ alkylene;
J represents C₂₋₄ alkylene;
Z represents a direct bond or C₁₋₃ alkylene;
R¹⁰ and R¹¹ independently represent H or C₁₋₄ alkyl;
n represents 0 or 2;
R⁴ represents phenyl or pyridyl, both of which groups are optionally substituted by one or more substituents selected from cyano, halo, nitro, C₁₋₆ alkyl, C₁₋₆ alkoxy, —NH₂, —C(O)N(R^{13e})R^{13f}, —N(R^{13g})C(O)R^{13h} and —N(R^{13m})S(O)₂—R^{12b};
R^{12b} represents C₁₋₃ alkyl;
R^{13e} to R^{13m} independently represent, at each occurrence when used herein, H or C₁₋₄ alkyl;
Het¹ to Het⁵ are optionally substituted by one or more substituents selected from ═O, cyano, halo, nitro, C₁₋₄ alkyl, C₁₋₄ alkoxy, —N(R^{14a})R^{14b}, —C(O)R^{14c} and C(O)OR^{14d};
R^{14a} to R^{14d} independently represent H, C₁₋₄ alkyl or aryl;
optional substituents on aryl and aryloxy groups, are unless otherwise stated, one or more substituents selected from cyano, halo, nitro, C₁₋₄ alkyl and C₁₋₄ alkoxy.

More preferred compounds of formula I include those in which R¹ represents an amino protective group or a structural fragment of formula Ia in which:

R² represents H, methyl, —OR⁵ or —N(H)R⁶;
R³ represents H or methyl;
R⁵ represents H, C₁₋₂ alkyl or phenyl (which phenyl group is optionally substituted by one or more substituents selected from cyano and C₁₋₄ alkoxy);
R⁶ represents H, C₁₋₂ alkyl, phenyl (which phenyl group is optionally substituted by one or more substituents selected from cyano, halo, nitro, C₁₋₄ alkyl and C₁₋₄ alkoxy), —C(O)—R^{8a} or —C(O)O—R^{8b};
R^{8a} and R^{8b} independently represent C₁₋₆ alkyl;
A represents C₁₋₄ alkylene;
B represents —Z—, —Z—N(R¹¹)—, —Z—S(O)₂— or —Z—O—;
R¹¹ represents H or methyl;
R⁴ represents pyridyl or phenyl, which latter group is optionally substituted by one to three substituents selected from cyano, nitro, C₁₋₂ alkoxy, NH₂ and —N(H)S(O)₂CH₃.

Yet more preferred compounds of formula I include those in which R¹ represents an amino protective group or a structural fragment of formula Ia in which:

R² represents H, —OR⁵ or —N(H)R⁶;
R⁵ represents H or phenyl (optionally substituted by one or more substituents selected from cyano and C₁₋₂ alkoxy);
R⁶ represents H, phenyl (optionally substituted by one or more cyano groups) or —C(O)O—C₁₋₅ alkyl;
A represents C₁₋₃ alkylene;
B represents —Z—, —Z—N(H)—, —Z—S(O)₂— or —Z—O—;
R⁴ represents phenyl, substituted by cyano in the ortho- and/or, in particular, the para-position relative to B.

Particularly preferred compounds of formula I include those in which R¹ represents an amino protective group or a structural fragment of formula Ia in which:

R² represents H or —OH;
R³ represents H;
A represents CH₂;
B represents —Z—, —Z—N(H)— or —Z—O;
Z represents a direct bond or C₁₋₂ alkylene;
R⁴ represents para-cyanophenyl.

Especially preferred compounds of formula I include those in which R¹ represents an amino protective group or one of the following sub-structures:

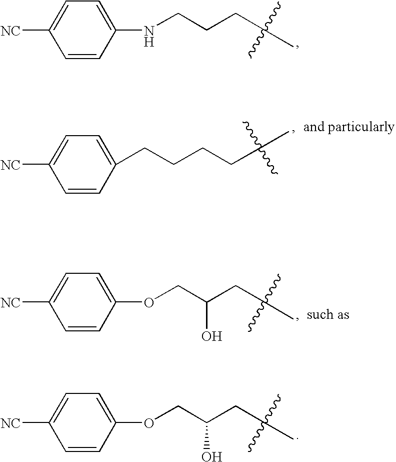

In an alternative embodiment of the invention, values of R¹ that may be mentioned include an amino protective group or one of the following sub-structures

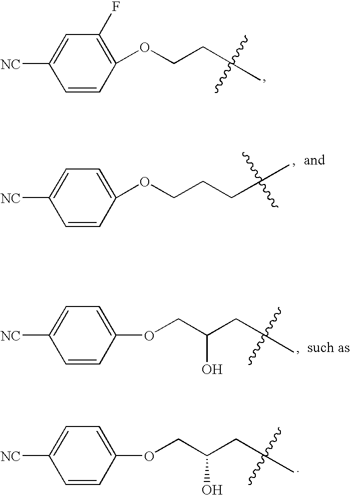

wherein R^{4a} represents F or, particularly, H and R^{10a} is as hereinbefore defined (e.g. H or, particularly, CH₃).

In another embodiment of the invention compounds of formula I that may be mentioned include those in which R¹ represents 2-phenethyl (optionally substituted in the phenyl part by one or more substituents (e.g. two substituents or, particularly, one substituent) selected from halo (e.g. chloro or, particularly, fluoro) and C₁₋₄ alkoxy (e.g. methoxy)). In a particular embodiment, however, the 2-phenethyl group is unsubstituted.

It is preferred that the process of the invention is carried out to provide compounds of formula I in which R¹ is an amino protective group.

Amino protective groups that may be mentioned, in particular with respect to R¹, include those which provide the carbamate, N-alkyl and N-sulfonyl groups mentioned hereinbefore. Specific amino protective groups that R¹ may represent thus include tert-butoxycarbonyl (to form a tert-butylcarbamate group), 3,4-dimethoxybenzyl, o-nitrobenzyl, benzyl and, particularly, a benzenesulfonyl group (which latter group is optionally substituted by one or more substituents mentioned hereinbefore with respect to substituents on an aryl group). Such benzenesulfonyl groups include 4-nitrobenzenesulfonyl, 2,4-dinitrobenzenesulfonyl, 2- or 4-fluorobenzenesulfonyl, 2- or 4-chlorobenzenesulfonyl, 4-bromobenzenesulfonyl, 4-methylbenzenesulfonyl, 4-methoxybenzenesulfonyl, 2,4,6-trimethylbenzenesulfonyl and, especially, unsubstituted benzenesulfonyl groups.

Preferred compounds of formula III include those in which L¹ represents halo, arenesulfonate, perfluoroalkanesulfonate or alkanesulfonate (e.g. p-toluenesulfonate, 2- or 4-nitrobenzenesulfonate, methanesulfonate, benzenesulfonate or trifluoromethanesulfonate). Particularly preferred compounds of formula III include those in which L¹ represents halo (especially chloro).

It is preferred that the compound of formula III is employed in the form of a single enantiomer, or in enantiomerically enriched form. For example, when L¹ represents chloro, the compound of formula III (epichlorohydrin) is preferably employed in the (S)- or, particularly, the (R)-enantiomeric form.

The aqueous solvent system employed in the process of the invention may be water, or water mixed with organic solvent that is miscible with water. In this respect, organic solvents that may be mentioned include tetrahydrofuran and C₁₋₄ alkyl alcohols (such as methanol, ethanol and IMS). When a water-miscible organic solvent is employed, then that solvent is preferably a C₁₋₄ alkyl alcohol (e.g. IMS). However, the most preferred aqueous solvent system is water on its own (i.e. not mixed with any organic solvents).

The stoichiometric ratio of the compound of formula II to the compound of formula III is at least 1:2, but is preferably any ratio from 1:2 to 1:8. Particularly preferred ratios include those from 1:2 to 1:6, such as 1:4 or thereabouts.

It is preferred that compounds of formulae II and III are mixed with the aqueous solvent system prior to the introduction of base.

The reaction is preferably performed at any temperature from 30 to 100° C., such as from 35 to 60° C. (e.g. from 40 to 55° C.). It is further preferred that the mixture of compounds of formulae II and III and the aqueous solvent system is raised to the specified temperature prior to the introduction of base.

As stated above, the pH of the reaction mixture is raised to between 10 and 13 (e.g. between 11.0 and 12.5, such as between 11.5 and 12.0) during a first period of base addition and is then, during a second period of base addition, controlled such that it is maintained within that pH range. During the second period of base addition, the pH is preferably maintained within the specified range by controlling the rate of base addition.

As also stated above, the time ratio of the first to the second period of base addition is 1:5 or less. Preferably, this ratio is 1:8 or less, such as 1:10 or less or, particularly, 1:12 or less. For example, when the reaction is performed on a laboratory scale (e.g. employing about 0.75 moles of a compound of formula II), then the ratio of first to second periods of base addition is preferably 1:20 or less, such as 1:30 or less (e.g. between 1:36 and 1:48). Further, when the reaction is performed on a plant scale (e.g. employing about 1 kmole of a compound of formula II), then the ratio preferably has a value from 1:5 to 1:10.

It is preferred that the reaction is maintained at the specified pH until it is substantially complete (e.g. until the point where 95% or more of the compound of formula II has been consumed).

The base employed in the process of the invention is preferably a water-soluble base. Bases that may be mentioned therefore include alkali metal carbonates, alkali metal hydrogencarbonates and/or, particularly, alkali metal hydroxides (e.g. sodium hydroxide).

The base may be employed as a solid or, preferably, in the form of an aqueous solution. When the base is added as an aqueous solution, the percentage weight of the base in water is between 5 and 50, preferably between 20 and 40, such as about 31% w/w.

During the first period of base addition, the temperature of the reaction mixture may rise. It is preferred that the rate of base addition during the first period is such as to maintain the temperature of the reaction mixture in the range from 30 to 65° C., preferably from 35 to 60° C. (e.g. from 40 to 55° C.).

Unless otherwise stated, when molar equivalents and stoichiometric ratios are to quoted herein with respect to acids and bases, these assume the use of acids and bases that provide or accept only one mole of hydrogen ions per mole of acid or base, respectively. The use of acids and bases having the ability to donate or accept more than one mole of hydrogen ions is contemplated and requires corresponding recalculation of the quoted molar equivalents and stoichiometric ratios. Thus, for example, where the acid employed is diprotic, then only half the molar equivalents will be required compared to when a monoprotic acid is employed. Similarly, the use of a dibasic compound (e.g. Na₂CO₃) requires only half the molar quantity of base to be employed compared to what is necessary where a monobasic compound (e.g. NaHCO₃) is used, and so on.

When the reaction between compounds of formulae II and III is substantially complete, the product may be isolated by techniques known to those skilled in the art, such as evaporation of solvent and any excess volatile reagents that may be employed, extraction with a suitable organic solvent, filtration and/or crystallisation.

Suitable organic solvents that may be employed for extraction of a compound of formula I include those that are immiscible with water, such as di(C₁₋₆ alkyl)ethers (such as di(C₁₋₄ alkyl)ethers, e.g. diethyl ether), C₁₋₆ alkyl acetates (such as C₁₋₄ alkyl acetates, e.g. ethyl acetate), higher alkyl (e.g. C₆₋₁₀) alcohols, chlorinated hydrocarbons (e.g. chlorinated C₁₋₄ alkanes such as dichloromethane, chloroform and carbon tetrachloride), hexane, petroleum ether, and aromatic hydrocarbons, such as benzene, chlorobenzene and mono-, di- or tri-alkylbenzenes (e.g. mesitylene, xylene, or toluene). Preferred organic solvents for extraction include chlorobenzene.

The product may, if desired, be further purified using techniques known to those skilled in the art (e.g. by chromatography, distillation and/or recrystallisation).

Compounds of formula II and III and derivatives thereof, are either commercially available, are known in the literature (see, for example, international patent applications WO 01/28992, WO 02/83690 and WO 02/28864, the disclosures of which are hereby incorporated by reference) or may be obtained by conventional synthetic procedures, in accordance with known techniques, from readily available starting materials using appropriate reagents and reaction conditions.

As stated above, compounds of formula I may be isolated and, if desired, further purified using techniques known to those skilled in the art.

However, in a preferred embodiment of the present invention, compounds of formula I are further elaborated to provide 3,7-dihydroxy-1,5-diazacyclooctanes.

Thus according to a second aspect of the invention, there is provided a process for the preparation of a compound of formula IV,

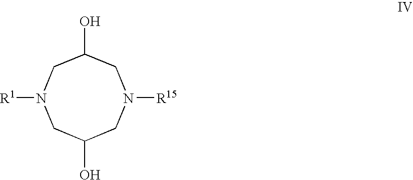

or a salt and/or solvate thereof;
wherein R¹⁵ represents H, an amino protective group or a structural fragment of formula Ia, as defined above; and
R¹ is as defined above;
which process comprises reaction of a compound of formula I, as defined above, with a compound of formula V,

H₂N—R¹⁵ V

or a salt and/or solvate thereof,
wherein R¹⁵ is as defined above; and
wherein the compounds of formula I and V are added, separately, simultaneously and at a substantially equivalent rate of moles per minute, to a reaction vessel containing solvent.

In a preferred embodiment of the process according to the second aspect of the invention, the compound of formula I is produced using the process according to the first aspect of the invention.

In this respect, and according to a third aspect of the invention, there is provided a process for the preparation of a compound of formula IV, as hereinbefore defined, or a salt and/or solvate thereof, which process comprises:
(i) a process according to the first aspect of the invention, as hereinbefore described, for the preparation of a compound of formula I, as hereinbefore defined; and then
(ii) reaction of the resulting compound of formula I, or a salt and/or solvate thereof, with a compound of formula V, as hereinbefore defined, wherein the compounds of formulae I and V are added, separately, simultaneously and at a substantially equivalent rate of moles per minute, to a reaction vessel containing solvent.

Preferred compounds of formula IV include those in which:

R¹ and R¹⁵ do not both represent a structural fragment of formula Ia;
R¹ takes the values indicated hereinbefore as preferred with respect to compounds of formula I;
R¹⁵ represents an amino protective group.

In an alternative embodiment of the invention, compounds of formula IV that may be mentioned include those in which one of R¹ and R¹⁵ represents 2-phenethyl and the other represents an amino protective group (e.g. R¹⁵ represents 2-phenethyl and R¹ represents an amino protective group such as benzenesulfonyl or benzyl, or R¹ represents 2-phenethyl and R¹⁵ represents an amino protective group such as benzenesulfonyl or benzyl). In this embodiment, the 2-phenethyl group may be optionally substituted as described above in relation to compounds of formula I.

Amino protective groups that R¹⁵ may represent include those that provide the N-alkyl groups mentioned hereinbefore. Particular amino protective groups that may be mentioned with respect to R¹⁵ thus include 3,4-dimethoxybenzyl, o-nitrobenzyl and, especially, benzyl groups.

When R¹ and R¹⁵ both represent amino protective groups, then it is preferred (though it is not necessary in all cases) that the two groups are orthogonal. For example, when R¹ represents an acid-labile amino protective group, then R¹⁵ represents an N-alkyl group, such as those mentioned hereinbefore with respect to R¹⁵ (e.g. benzyl). In this way, the group that R¹ represents may be cleaved under conditions (e.g. acid-catalysed hydrolysis) to which the R¹⁵ group is resistant.

The term “acid-labile amino protective group”, when used herein includes references to optionally substituted benzenesulfonyl groups defined hereinbefore with respect to R¹ (e.g. 2- or 4-fluorobenzene-sulfonyl, 2- or 4-chlorobenzenesulfonyl, 4-bromobenzenesulfonyl, 4-methylbenzenesulfonyl, 4-methoxybenzenesulfonyl, 2,4,6-trimethylbenzenesulfonyl and, especially, unsubstituted benzenesulfonyl).

In the processes according to the second and third aspects of the invention, it is preferred that the stoichiometric ratio of the compound of formula I to the compound of formula V is in the range of 2:1 to 1:2, such as about 1:1 (e.g. 10:9).

Further, solvents that may be present in the vessel utilised for the reaction between the compounds of formulae I and V include the water-miscible organic solvents described hereinbefore. In this respect, solvents that may be mentioned include C₁₋₄ alkyl alcohols, such as ethanol and, particularly, IMS or methanol.

The reaction between the compounds of formulae I and V may be performed at ambient or, preferably, elevated temperature (e.g. at reflux). Additionally, the temperature at which the reaction is be performed may, for any given solvent, be increased above the reflux temperature at atmospheric pressure by utilisation of elevated pressure (e.g. from 0.1 to 2 atmospheres of overpressure). The elevated pressure may be generated, for example, by heating, in a sealed vessel, the reaction mixture from ambient temperature to the chosen reaction temperature. For example, the reaction may be conducted at from 60 to 90° C. (e.g. 78 or 88° C.) under 0.4 to 0.8 atmospheres (e.g. about 0.5 atmospheres) of overpressure.

In the process according to the third aspect of the invention, the compound of formula I is preferably employed directly (i.e. without isolation) in the form in which it is obtained from performing the process according to the first aspect of the invention. For example, where the compound of formula I is extracted into an organic solvent after being formed (i.e. after reaction between the compounds of formula II and III is complete), then the compound of formula I may be employed in step (b) of the process according to the third aspect of the invention as a solution in that organic solvent (e.g. chlorobenzene).

As stated above, in the processes according to the second and third aspects of the invention, the compounds of formulae I and V are added simultaneously and separately to the reaction vessel.

By “simultaneously” we include references to the compounds of formulae I and V having their addition to the reaction vessel both initiated and terminated at approximately the same time.

Further, by “separately” we include references to the compounds of formulae I and V being kept physically separate (e.g. as separate solutions or stores of neat compound) until the moment of their addition to the reaction vessel.

The rates of moles per minute at which the compounds of formulae I and V are added to the reaction vessel can vary greatly, depending upon the scale upon which the reaction is performed. Thus, the rate of addition might vary form 0.1 millimole per minute to 10 moles per minute (e.g. about 1 mmol/min to about 3 moles/min). However, it is preferred that the rate of addition is measured relative to the volume of solvent initially present in the reaction vessel (i.e. in mole/min.L) and that this rate is in the region of 0.1 to 10 mmol/min.L (e.g. 0.5 to 1.5, such as from 0.9 to 0.95 mmol/min.L).

The compounds of formulae I and V may be added to the reaction vessel in either a portion-wise or, particularly, continuous manner. The addition may be achieved using means known to those skilled in the art, such as by metered (e.g. syringe) pumps or rotameters.

When the reaction is substantially complete, the product may be isolated and, if desired, further purified as hereinbefore described.

Compounds of formula IV may be further elaborated to provide oxabispidines by dehydrative cyclisation.

Thus, according to a fourth aspect of the invention, there is provided a process for the preparation of a compound of formula VI,

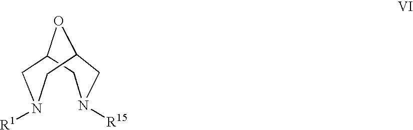

or a salt and/or solvate thereof;
wherein R¹ and R¹⁵ are as hereinbefore defined;
which process comprises a process as hereinbefore described for the preparation of a compound of formula IV, followed by dehydrative cyclisation of that compound.

Preferred compounds of formula VI include those in which R¹ and R¹⁵ represent the preferred values mentioned above in respect of compounds of formula IV.

In another embodiment of the invention, compounds of formula VI that may be mentioned include those in which one of R¹ and R¹⁵ represents 2-phenethyl and the other represents an amino protective group (e.g. R¹⁵ represents 2-phenethyl and R¹ represents an amino protective group such as benzenesulfonyl or benzyl, or R¹ represents 2-phenethyl and R¹⁵ represents an amino protective group such as benzenesulfonyl or benzyl). In this embodiment, the 2-phenethyl group may be optionally substituted as described above in relation to compounds of formula I.

The process according to the fourth aspect of the invention may be performed under reaction conditions known to the skilled person, such as those described in WO 02/28864 and WO 02/83690. Conditions thus include reaction in the presence of a suitable dehydrating reagent, such as sulfuric acid (e.g. concentrated sulfuric acid) or a sulfonic acid (e.g. an alkane or perfluoroalkanesulfonic acid, such as methanesulfonic acid, including anhydrous methanesulfonic acid).

The skilled person will appreciate that certain R¹ and/or R¹⁵ groups of compounds of formulae IV and VI may be removed or converted into other R¹ and/or R¹⁵ groups respectively. For example, compounds wherein R¹⁵ represents an amino protective group may be converted to corresponding compounds wherein R¹⁵ represents H by cleavage of that amino protective group. However, in a particularly convenient process, a compound of formula VI in which R¹ represents an amino protective group is further elaborated by removal of that amino protective group.

Thus, according to a fifth aspect of the invention, there is provided a process for the preparation of a compound of formula VII,

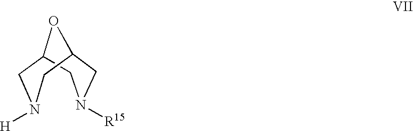

or a salt and/or solvate thereof;
wherein R¹⁵ is as hereinbefore defined, which process comprises a process as hereinbefore described for the preparation of a compound of formula VI in which R¹ represents an amino protective group, followed by removal of that protective group.

Preferred compounds of formula VII include those in which R¹⁵ represents an amino protective group, such as benzyl.

In an alternative embodiment of the invention, compounds of formula VII that may be mentioned include those in which R¹⁵ represents 2-phenethyl. In this embodiment, the 2-phenethyl group may be optionally substituted as described above in relation to compounds of formula I.

Methods of deprotection of an amino protective groups that R¹ may represent are known to those skilled in the art and include methods disclosed in the reference “Protective Groups in Organic Synthesis”, 3^{rd} edition, T. W. Greene & P. G. M. Wutz, Wiley-Interscience (1999). For example, when R¹ represents an acid-labile amino protective group, such as an unsubstituted benzenesulfonyl group, then conditions that may be employed include those described in WO 02/83690 and WO 02/28864 (e.g. reaction in the presence of an acid (e.g. concentrated hydrobromic acid) at, for example, elevated temperature (e.g. 95° C. or, if a slight overpressure (e.g. about 0.2 to 0.27 atmospheres (3 to 4 psi)) is utilised in the reaction, from 100 to 125° C., such as about 122° C.).

Alternatively, and according to a particularly preferred mode of performing the present invention, the removal of the R¹ group, when that group represents a benzenesulfonyl group, is effected by hydrolysis in sulfuric acid. In this respect, and according to sixth and seventh aspects of the invention, there is provided:
(I) a process for the preparation of a compound of formula VII, as hereinbefore defined, or a salt and/or solvate thereof, which process comprises sulfuric acid-catalysed hydrolysis of a compound of formula VIa,

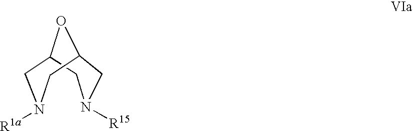

or a salt and/or solvate thereof, wherein R^{1a} represents benzenesulfonyl optionally substituted on the benzene ring by one or more substituents selected from C₁₋₄ alkyl, C₁₋₄ alkoxy and halo, and R¹⁵ is as hereinbefore defined; and
(II) a process for the preparation of a compound of formula VII, or a salt and/or solvate thereof, as hereinbefore defined, which process comprises
   (a) a process as hereinbefore described for the preparation of a compound of formula VI in which R¹ represents R^{1a}, as hereinbefore defined, followed by
   (b) reaction of the resulting compound of formula VI, or a salt and/or solvate thereof, with concentrated sulfuric acid.

Specific values of R^{1a} that may be mentioned include 2- or 4-fluorobenzenesulfonyl, 2- or 4-chlorobenzenesulfonyl, 4-bromobenzenesulfonyl, 4-methylbenzenesulfonyl, 4-methoxybenzenesulfonyl, 2,4,6-trimethylbenzenesulfonyl and, particularly, unsubstituted benzenesulfonyl.

Hydrolytic cleavage of the R^{1a} group in the processes according to the sixth and seventh aspects of the invention may is effected by utilising sulfuric acid, and preferably concentrated sulfuric acid. Further, the hydrolysis is preferably performed by heating a mixture of a compound of formula VIa and concentrated sulfuric acid to elevated temperature (e.g. to 100° C. or above, such as to a temperature from 100 to 135° C. or, particularly, to 130° C. or thereabouts).

As the skilled person will appreciate from the foregoing, the same reagent (i.e. sulfuric acid) may be used to effect dehydrative cyclisation of compounds of formula IV, as well as removal of the R^{1a} group of resulting compounds of formula VIa. In this respect, and according to eighth and ninth aspects of the invention, there is provided:
(A) a process for the preparation of a compound of formula VII, or a salt and/or solvate thereof, as hereinbefore defined, which process comprises reaction of a compound of formula IVa,

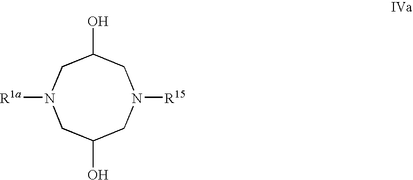

or a salt and/or solvate thereof, wherein R^{1a} and R¹⁵ are as hereinbefore defined, with concentrated sulfuric acid;
(B) a process for the preparation of a compound of formula VII, or a salt and/or solvate thereof, as hereinbefore defined, which process comprises
   (a) a process as hereinbefore described for the preparation of a compound of formula IV in which R¹ represents R^{1a}, as hereinbefore defined, followed by
   (b) reaction of the resulting compound of formula IV, or a salt and/or solvate thereof, with concentrated sulfuric acid.

Those skilled in the art will appreciate that compounds of formula IV in which R¹ represents R^{1a} (i.e. those mentioned in process (B) above) are compounds of formula IVa.

Further, those skilled in the art will also appreciate that the processes according to the eighth and ninth aspects of the invention are, or include, a “one-pot” procedure, wherein the concentrated sulfuric acid first effects dehydrative cyclisation of the compound of formula IVa (to provide an intermediate compound of formula VIa, as hereinbefore defined, which intermediate is not isolated) and then catalyses hydrolysis of the R^{1a} (N-benzenesulfonyl) group, so as to provide the target compound of formula VII.

Salts of the compounds of formulae IV, V, VI and VII that may be mentioned include acid addition salts. Solvates that may be mentioned include hydrates.

As stated above, the “one-pot” dehydrative cyclisation and deprotection reaction of the processes according to the eighth and ninth aspects of the invention is effected by concentrated sulfuric acid. When used herein, the term “concentrated sulfuric acid” refers to an aqueous mixture having a H₂SO₄ content of more than 40% by weight (such as more than 50, 60, 70, 75 or, particularly, 80% H₂SO₄ by weight).

When reacted with sulfuric acid, the compounds of formulae IV, IVa and VIa may either be added to sulfuric acid or, vice versa. In one embodiment of the invention, the compound of formula IV, IVa or VIa is added to sulfuric acid that is at a temperature above 80° C. (e.g. 100° C. or above).

Compounds of formula VII may be further elaborated to provide oxabispidines having different N-substituents.

Thus, according to a tenth aspect of the invention, there is provided a process for the preparation of a compound of formula VIII,

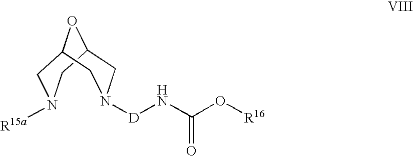

wherein R^{15a} represents R¹⁵, as hereinbefore defined, except that it does not represent H;
D represents C₂₋₆ n-alkylene; and
R¹⁶ represents C₁₋₆ alkyl (optionally substituted by one or more substituents selected from —OH, halo, cyano, nitro and aryl) or aryl,
which process comprises a process as described hereinbefore for the preparation of a compound of formula VII in which R¹⁵ is other than H, followed by reaction, in an organic solvent (e.g. toluene), of that compound with a compound of formula IX,

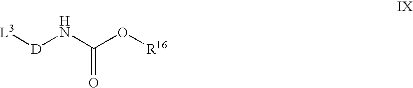

wherein L³ represents a suitable leaving group (e.g. halo or, particularly R¹⁷—S(O)₂—O—, in which R¹⁷ represents unsubstituted C₁₋₄ alkyl, C₁₋₄ perfluoroalkyl or phenyl, which latter group is optionally substituted by one or more substituents selected from C₁₋₆ alkyl, halo, nitro and C₁₋₆ alkoxy) and D and R¹⁶ are as hereinbefore defined.

This reaction may be performed under conditions known to those skilled in the art, such as those described in WO 02/83690 (such as at elevated temperature (e.g. 68° C.).

Values of D that may be mentioned in relation to compounds of formulae VIII and IX include —(CH₂)₃— and, particularly, —(CH₂)₂—.

Preferred compounds of formula VIII include those in which:

R^{15a} represents an amino piotective group, such as benzyl;
R¹⁶ represents C₁₋₆ alkyl (e.g. saturated C₁₋₆ alkyl) and, preferably, saturated C₃₋₅ alkyl (e.g. saturated C₄ alkyl), such as tert-butyl.

Preferred compounds of formula IX include those in which R¹⁷ represents phenyl, optionally substituted by one or more (e.g. one to three) substituents (e.g. one substituent) selected from C₁₋₃ alkyl (e.g. methyl), halo and nitro, particularly unsubstituted phenyl, methylphenyl (such as 4-methylphenyl) or trimethylphenyl (such as 2,4,6-trimethylphenyl).

Compounds of formula VIII in which R^{15a} represents an amino protective group may also be further elaborated.

Thus, according to eleventh and twelfth aspects of the invention, there is provided the following.

(A) A process for the preparation of a compound of formula X,

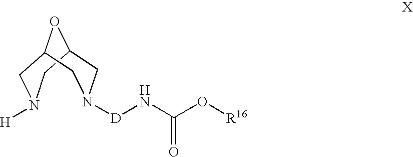

wherein D and R¹⁶ are as hereinbefore defined, which process comprises a process as hereinbefore described for the preparation of a compound of formula VIII in which R^{15a} represents an amino protective group, followed by removal of that protective group.
   (B) A process for the preparation of a compound of formula XI,

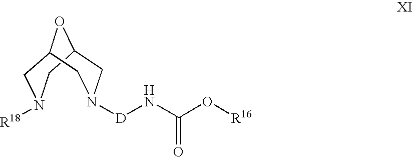

or a pharmaceutically acceptable derivative thereof;
wherein R¹⁸ represents a structural fragment of formula Ia, as hereinbefore defined, and D and R¹⁶ are as hereinbefore defined,
which process comprises a process as defined in (A) above for the preparation of a corresponding compound of formula X, followed by reaction of that compound with,
1) a compound of formula XII,

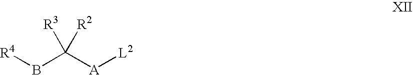

wherein L² represents a leaving group (e.g. mesylate, tosylate, mesitylenesulfonate, or halo) and R², R³, R⁴, A and B are as hereinbefore defined,
2) for compounds of formula XI in which A represents C₂ alkylene and R² and R³ together represent ═O, a compound of formula XIII,

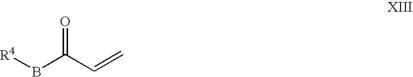

wherein R⁴ and B are as hereinbefore defined, or
3) for compounds of formula XI in which A represents CH₂ and R² represents —OH or —N(H)R⁶, a compound of formula XIV,

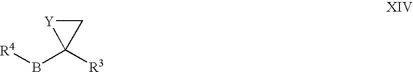

wherein Y represents —O— or —N(R⁶)— and R³, R⁴, R⁶ and B are as hereinbefore defined.

Pharmaceutically acceptable derivatives of the compound of formula XI include salts (e.g. acid addition salts) and solvates.

Pharmaceutically acceptable derivatives of the compounds of formula XI also include, at the oxabispidine or (when R⁴ represents pyridyl)pyridyl nitrogens, C₁₋₄ alkyl quaternary ammonium salts and N-oxides, provided that when a N-oxide is present:
(a) no Het (Het¹, Het², Het³, Het⁴ and Het⁵) group contains an unoxidised S-atom; and/or
(b) n does not represent 0 when B represents —Z—S(O)ₙ—.

Values of D and R¹⁶ that may be mentioned in relation to compounds of formulae X and XI include those mentioned above in relation to compounds of formulae VIII and IX.

In the process according to the twelfth aspect of the invention (i.e. that outlined at (B) above), preferred compounds of formula XI include those in which R¹⁸ represents the preferred values of the structural fragment of formula Ia mentioned hereinbefore in respect of compounds of formula I.

Removal of amino protective group in the process according to the eleventh aspect of the invention, as well as the alkylation of the process according to the twelfth aspect of the invention (i.e. the reaction with a compound of formula XII, XIII or XIV) may be carried out under conditions known to the skilled person, such as those described in WO 02/83690 or WO 2004/035592.

For example, when the amino protective group that R^{15a} represents is benzyl, then that group may be removed by hydrogenation in the presence of an appropriate catalyst (e.g. Pd/C or Pt/C).

Further, reaction of the compound of formula X:
(a) with a compound of formula XII may, for example, be performed at elevated temperature (e.g. between 35° C. and reflux temperature) in the presence of a suitable base (e.g. triethylamine or potassium carbonate) and an appropriate solvent (e.g. ethanol, toluene or water (or mixtures thereof));
(b) with a compound of formula XIII may, for example, be performed at room temperature in the presence of a suitable organic solvent (e.g. ethanol); and
(c) with a compound of formula XIV may, for example, be performed at elevated temperature (e.g. between 60° C. and reflux) in the presence of a suitable solvent (e.g. water, iso-propanol, ethanol or toluene (or mixtures thereof)).

Compounds of formulae IX, XII, XIII and XIV, and derivatives thereof, are either commercially available, are known in the literature (e.g. described in WO 02/83690) or may be obtained by conventional synthetic procedures, in accordance with known techniques, from readily available starting materials using appropriate reagents and reaction conditions.

In addition to these further aspects of the invention described above, the skilled person will appreciate that certain compounds of formula XI may be prepared from certain other compounds of formula XI, or from structurally related compounds. For example, compounds of formula XI in which R¹⁸ represents certain structural fragments of formula Ia may be prepared, in accordance with relevant processes known in the art, by the respective interconversion of corresponding compounds of formula XI in which R¹⁸ represents other structural fragments of formula Ia (for example by analogy with the processes described in international patent application numbers WO 99/31100, WO 00/76997, WO 00/76998, WO 00/76999, WO 00/77000 and WO 01/28992).

It will be appreciated by those skilled in the art that, in the processes described above, the functional groups of intermediate compounds may be, or may need to be, protected by protecting groups.

In any event, functional groups which it is desirable to protect include hydroxy and amino. Suitable protecting groups for hydroxy include trialkylsilyl and diarylalkylsilyl groups (e.g. tert-butyldimethylsilyl, tert-butyldiphenylsilyl or trimethylsilyl), tetrahydropyranyl and alkylcarbonyl groups (e.g. methyl- and ethylcarbonyl groups). Suitable protecting groups for amino include the amino protective groups mentioned hereinbefore, such as benzyl, sulfonyl (e.g. benzenesulfonyl or 4-nitrobenzenesulfonyl), tert-butyloxycarbonyl, 9-fluorenylmethoxycarbonyl or benzyloxycarbonyl.

The protection and deprotection of functional groups may take place before or after any of the reaction steps described hereinbefore.

Protecting groups may be removed in accordance with techniques which are well known to those skilled in the art and as described hereinafter.

The use of protecting groups is described in “Protective Groups in Organic Chemistry”, edited by J. W. F. McOmie, Plenum Press (1973), and “Protective Groups in Organic Synthesis”, 3^{rd} edition, T. W. Greene & P. G. M. Wutz, Wiley-Interscience (1999).

The processes of the invention may have the advantage that compounds of formulae I, IV, VI and VII may be prepared in higher yields, in higher purity, by way of fewer steps (i.e. involving fewer unit operations), in less time, in a more convenient form (e.g. in a form that is easier to handle), from more convenient (e.g. easy to handle) precursors, at a lower cost and/or with less usage and/or wastage of materials (including reagents and solvents) compared to the procedures disclosed in the prior art.

“Substantially”, when used herein, may mean at least greater than 50%, preferably greater than 75%, for example greater then 95%, and particularly greater than 99%.

The term “volume” (vol.) or “relative volume” (rel. vol.), when used herein, refers to the volume (in milliliters) per gram of reagent employed.

The invention is exemplified, but in no way limited, by the following examples.

### EXAMPLE 1

### Chirally enriched N,N-Bis(2(R)-oxiranylmethyl)benzenesulfonamide

### Alternative 1

Benzenesulfonamide (120 g, 0.763 moles), (R)-epichlorohydrin (282.6 g, 3.054 moles) and water (960 g) were added to a 2 L reaction flask. The mixture was heated to 40° C. and then sufficient sodium hydroxide solution (31%) was added over approximately 5 mins such that the pH was raised to 11.5-12.0 (in an alternative procedure, 25% sodium hydroxide solution can be employed). The remainder of the sodium hydroxide (201 g, 1.557 moles in total) was then added at such a rate as to maintain the pH at 11.5-12.0 and the temperature at 40-50° C. (usually requires addition over 3-4 hours). The reaction mixture was then stirred for 2 hours at 40-45° C. and distilled to remove 3 volumes (360 mL) of water/epichlorohydrin at 50 mbar (5 kPa) with a maximum contents (source vessel) temperature of 43° C. Chlorobenzene was then added (221.4 g, 1.67 volumes) and the mixture was stirred for 0.5 hours before being allowed to settle. The lower product (chlorobenzene) layer was separated and the extraction process repeated using a further portion of chlorobenzene (44.3 g, 0.33 vols.). The two product layers were combined for use in the next step (see Example 2, Alternative 1 below).

### Alternative 2

Benzenesulfonamide (175 kg, 1 eq.), water (1365 kg, 8 rel. vol.) and (R)-epichlorohydrin (412 kg, 4 eq.) were charged to a reaction vessel. The reactants were heated to 40° C. Sufficient aqueous sodium hydroxide was added, over the course of approximately 20 minutes, to adjust the pH to 11.5-12.0. The remainder was then charged in a controlled manner over approximately 150 minutes, such that the temperature of the reaction was maintained between 40° C. and 50° C., and the pH remained in the range 11.5 to 12.0 (total charge: 90.8 kg in 202 kg of water). After the addition of sodium hydroxide was complete, the reaction was stirred between 40° C. and 45° C. for 2 hours. The excess (R)-epichlorohydrin was removed as a water azeotrope by vacuum distillation (ca. 60 mbar (6 kPa), internal temperature 43° C. maximum, 525 liters of distillate, 3 rel. vol.). Chlorobenzene (total of 387 kg, 2 rel. vol.) was then charged to the reaction in two portions. Following each addition, the mixture was stirred and then allowed to settle before the chlorobenzene layer was separated. The two chlorobenzene layers were then combined and used without further treatment in the next step (see Example 2, Alternative 2 below).

### EXAMPLE 2

### Chirally enriched 5-Benzyl-3(S),7(S)-dihydroxy-1-phenylsulfonyl-1,5-diazacyclooctane

### Alternative 1

Methanol (854 g, 18 volumes) was heated to reflux. Chirally enriched N,N-bis(2(R)-oxiranylmethyl)-benzenesulfonamide (0.382 mol; see Example 1, Alternative 1 above) and benzylamine (37.3 g, 0.347 moles) were concurrently added via syringe pumps over 6 hours into the reaction vessel at opposite sides of the reaction vessel. The reaction was maintained at reflux throughout the addition of the reagents. After addition was complete, the reaction solution was maintained at reflux for a further 3 hours before methanol (14 volumes, 840 mL) was distilled from the reaction vessel at atmospheric pressure. Chlorobenzene (266 g, 240 mL) was then added and the distillation continued until a further portion of methanol (4 volumes, 240 mL) had been collected from the reaction vessel. A second portion of chlorobenzene (133 g, 120 mL) was added and a mixture of solvent (4 volumes, 240 mL of a mixture of chlorobenzene/methanol) was distilled from the reaction mixture at 50 mbar (5 kPa). The remaining mixture (after the distillation) comprised the sub-title compound and chlorobenzene with a methanol content of <0.1% w/w. This solution was employed in the next step (see Example 3, Alternative 1 below).

### Alternative 2

Methanol (2494 kg, 18 rel. vol.—either fresh or recycled) was charged to a reaction vessel and heated to reflux temperature (approx. 65° C.). Simultaneously, and over approximately 6 hours were charged the chlorobenzene solution (containing chirally enriched N,N-bis(2(R)-oxiranylmethyl)benzenesulfonamide) from Example 1, Alternative 2 above and benzylamine (109 kg, 0.91 eq.). The batch was maintained at reflux throughout the addition. The reaction was stirred at approximately 65° C. (reflux temperature) for a further 3 hours. Methanol (1938 kg, 14 rel. vol.) was then removed by distillation at atmospheric pressure before chlorobenzene (775 kg, 4 rel. vol.) was added. The resulting solution was used without further treatment in the next step (see Example 3, Alternative 2 below).

### EXAMPLE 3

### 3-Benzyl-7-(phenylsulfonyl)-9-oxa-3,7-diazabicyclo[3.3.1]nonane

### Alternative 1

Chlorobenzene (598 g, 9 volumes) and water (7.2 g, 0.4 moles) were added to a solution of chirally enriched 5-benzyl-3(S),7(S)-dihydroxy-1-phenylsulfonyl-1,5-diazacyclooctane in chlorobenzene (0.382 moles; see Example 2, Alternative 1 above) and heated to 75° C. Sulfuric acid (98%, 134 g, 1.337 moles) was then added over 1 hour, whilst maintaining the temperature in the range 75-90° C. (In an alternative embodiment, chirally enriched 5-benzyl-3(S),7(S)-dihydroxy-1-phenylsulfonyl-1,5-diazacyclooctane may be added to sulfuric acid.) The biphasic reaction mixture was heated to 95° C. and stirred for 3 hours. The temperature was adjusted to 50° C. and methanol (57 g, 1.2 volumes) was added at such a rate as to maintain the temperature at between 50 and 60° C. The reaction mixture was basified by adding aqueous ammonia (17.5%, 346 g, 372 mL) over 2 hours at between 60 and 70° C., and then allowed to settle after 15 min of stirring (the mixture is kept at 60° C. during the period in which it is allowed to settle). The lower aqueous layer was separated and the upper organic layer transferred to the crystallising vessel. The aqueous layer was returned to the reaction vessel and the temperature was adjusted to 45° C. before chlorobenzene (133 g, 120 mL) was added. The separation process was repeated (i.e. the aqueous layer extracted and the phases separated) and the second organic phase combined with the first organic phase in the crystallising vessel. Chlorobenzene was then distilled (660 mL, 11 volumes) from the product layer at 50 mbar (5 kPa) and then methanol (470 g, 594 mL) was added over the course of 1 hour. The temperature was allowed to fall during this addition, after which the resulting slurry was cooled to 5° C. and held at that temperature for 1 hour before being filtered. The filter cake was then washed with two portions of methanol (2×47.4 g, (2×60 mL)), at either 5° C. or ambient temperature, and then suction dried for 30 mins. The product was transferred to a vacuum oven and dried to constant weight at 40° C. to provide the sub-title compound (yield 31% (42.5 g) over Alternative 1 of Examples 1, 2 and 3).

### Alternative 2

The chlorobenzene/methanol solution from Example 2, Alternative 2 above was distilled further at atmospheric pressure (removing a total of 700 liters (4 rel. vol.) of solvent). Fresh and/or recycled chlorobenzene (350 liters, 2 vols.) was charged, then distillation was continued under vacuum (ca. 50 mbar (5 kPa)) to complete solvent exchange to chlorobenzene (a total of a further 700 liters (4 rel. vol.) being removed through distillation). Further chlorobenzene (fresh or recovered, 1575 liters, 9 rel. vol.) and water (21 kg, 1.05 eq.) were charged and the batch heated to 75° C. Sulfuric acid (382 kg, 3.5 eq. of 98%) was charged over approximately 1 hour, whilst allowing the temperature to rise up to 90° C. The biphasic reaction mixture was maintained for a further 3 hours at 95° C. After cooling to 50-55° C., methanol (160 kg, 1.2 rel. vol.) was charged, whilst maintaining the temperature at 50-55° C. Aqueous ammonia (176 kg in 830 kg of water) was charged in a controlled manner whilst maintaining the contents at between 60-70° C. After stirring for 15 minutes, the batch was settled for 30 minutes and the layers separated. After back extracting the aqueous layer with chlorobenzene (388 kg, 2 vol.) the organic layers were combined and a total of 1925 liters (11 rel. vol.) of chlorobenzene were distilled under vacuum (50 mbar (5 kPa), 45° C.). Methanol (1330 kg, 9.6 rel. vol) was charged to the residue. The resultant slurry was cooled to 5° C., stirred for 1 hour, then the solids were isolated by filtration. The wet filter cake was dried under vacuum (˜50 mbar (5 kPa), 40° C. maximum temperature) to afford 130.5 kg of the title compound (32.7% yield over Alternative 2 of Examples 1, 2 and 3).

### EXAMPLE 4

### 3-Benzyl-9-oxa-3,7-diazabicyclo[3.3.1]nonane dihydrochloride

### Alternative 1

3-Benzyl-7-(phenylsulfonyl)-9-oxa-3,7-diazabicyclo[3.3.1]nonane (40 g, 0.112 mol; see Example 3, Alternative 1 above) and hydrobromic acid (48%, 179 g, 120 mL) were heated to 122° C. and stirred for 9 hours. The solution was cooled to 20° C. before toluene (173 g, 200 mL) was added and the resulting biphasic mixture stirred for 30 mins. After being allowed to settle, the lower aqueous layer of the biphasic mixture was separated and the upper toluene layer discarded. The aqueous layer was returned to the reaction vessel and sodium hydroxide (31%, 181 g, 141 mL) was added over 45 mins, allowing the temperature to rise to a maximum of 60° C. Toluene (156 g, 180 mL) was added and the temperature adjusted to 60° C. before the layers were separated and the lower aqueous layer discarded. The toluene layer, containing the product, was washed with water (120 g) at 60° C. before being cooled to 40° C., after which isopropanol (345 g, 440 mL) was added. Hydrochloric acid (36%, 25.9 g, 0.256 mol) was then added over 1 hour at 40-45° C., after which the mixture was cooled to 5° C. and stirred for 1 hour. The product was filtered, washed with iso-propanol (141 g, 180 mL) and then suction dried for 30 mins before being transferred to the vacuum oven and dried to constant weight at 40° C. (yield: 88%, 28.6 g).

### Alternative 2

3-Benzyl-7-(phenylsulfonyl)-9-oxa-3,7-diazabicyclo[3.3.1]nonane (107 kg, 1 eq.; see Example 3, Alternative 2 above) and hydrobromic acid (229 kg, 9.5 eq. in 248 kg water) were charged to a vessel, heated initially to 110 to 115° C. with the scrubber vent open, the vent was sealed then heating was continued under a slight positive pressure (4 psi (0.27 atmospheres)) to 122° C. then stirred for 9 hours at 122° C. After cooling to 15-20° C., toluene (463 kg, 5 rel. vol) was charged and the resulting biphasic mixture was stirred before being allowed to settle for 30 minutes. The layers were separated and the lower aqueous layer was returned to to the original vessel. To this vessel was then charged aqueous sodium hydroxide (149 kg, 12.5 eq. in 332 kg water) whilst maintaining the contents temperature below 80° C. The reaction mixture was cooled to a temperature in the range of 15 to 20° C. before toluene (416 kg, 4.5 rel. vol) was charged. The resulting biphasic mixture was heated to 60° C. and then stirred and settled for 30 minutes at 60° C. After separation, the toluene layer was washed with water (214 kg, 2 rel. vol.) at 60° C., then cooled to 15-20° C. Isopropyl alcohol (925 kg, 11 rel. vols.) was charged, and the contents adjusted to 40° C. Hydrochloric acid (25 kg, 2.3 eq. in 44.5 kg water) was charged in a controlled manner, keeping the contents in the range 40-45° C. After stirring for 1 hour at 40° C., the resultant slurry was cooled to 5° C. and stirring continued for a further 2 hours. The solids were isolated by filtration and dried (40° C. maximum temperature) to afford 78 kg (89.7%) of the title compound.

### Alternative 3

Water (72 mL) and concentrated sulfuric acid (228 mL) were added to 3-benzyl-7-(phenylsulfonyl)-9-oxa-3,7-diazabicyclo[3.3.1]nonane (100.06 g, 279 mmol; see Example 3 above). The reaction mixture was heated for 9 hours at 130° C., then left to cool to room temperature overnight. The acidic solution was poured into a clean vessel containing water (300 mL), and concentrated aqueous ammonia (35%) added dropwise over 2 hours (550 mL). After ammonia addition was complete, the pH of the reaction mixture was checked and found to be 10. Toluene (450 mL) was then added, and the temperature adjusted to 60° C. The lower (aqueous) layer was separated and discarded. To the remaining upper layers (organic layer and interfacial layer), 5 M sodium hydroxide solution (300 mL) was added. The mixture was re-heated to 60° C., and stirred for 15 minutes. The layers were separated and the lower aqueous phase removed. Isopropanol (1100 mL) was added to the organic phase and the resulting solution warmed to 43° C. Concentrated hydrochloric acid (54 mL) was then added over 1 hour, maintaining the temperature at between 40 and 45° C., which precipitated the product. The mixture was then cooled to 5° C., and stirred for 1 hour. The product was collected by filtration and the filter cake was washed by displacement with isopropanol (400 mL) before being dried as much as possible by suction (on the filter) and then in vacuo (for 64 hours at 40° C.). This gave the title compound as a crystalline, white solid (77.16 g, 95%).

### Alternative 4

77% sulfuric acid (126.3 g, 0.99 moles) and 98% sulfuric acid (68.4 g, 0.683 moles) are mixed carefully to afford 195 g of 85% sulfuric acid (1.675 moles, 15 eq.). (Alternatively, water and 98% sulfuric acid are mixed carefully to prepare the same quantity of 85% sulfuric acid.) The reaction mixture is heated to 100° C., before 3-benzyl-7-(phenylsulfonyl)-9-oxa-3,7-diazabicyclo-[3.3.1]nonane (40 g, 0.112 moles; see Example 3 above) is added, portion-wise, over the course of approximately 45 to 60 minutes. The reaction mixture is heated to 130° C., and stirred at this temperature for 9 hours. After the reaction mixture is cooled to 20 to 25° C., water (120 g) is added over the course of approximately 30 minutes, during which addition the reaction mixture is maintained at 20 to 50° C. At this point, 35% ammonia solution (193.6 g, 3.96 moles) is added over the course of approximately 2 hours, during which addition the reaction mixture is maintained at below 70° C. After verifying that the pH of the batch is 10 or above, toluene is added and the reaction mixture is stirred rapidly, at 70 to 75° C., for 15 minutes. The layers are allowed to settle for approximately 30 minutes, then the lower (aqueous) layer is discarded. To the remaining upper layers (organic layer and interfacial layer), is added 5M sodium hydroxide solution (139 g, 0.60 moles), and the reaction mixture is stirred for approximately 15 minutes at 60 to 65° C.

After settling for 30 minutes, the layers are separated, keeping any interfacial material with the aqueous layer. The product (toluene) layer is cooled to 40 to 45° C. before isopropanol (345 g, 440 mL) is added, followed by, over the course of 1 hour and at 40 to 45° C., 36% hydrochloric acid (26.0 g, 0.257 mols). The resulting mixture is cooled to 5° C. and stirred at this temperature for 1 hour. The product is isolated by filtration, washed with isopropanol (126 g, 160 mL) and then dried by suction (on the filter) for 30 mins, before being transferred to a vacuum oven. The title compound is then dried to constant weight at 40° C. (30.1 g, 92.5%).

### EXAMPLE 5

### 3-Benzyl-9-oxa-3,7-diazabicyclo[3.3.1]nonane

Water (11.2 mL) and concentrated sulfuric acid (24.5 mL) were added to 5-benzyl-3,7-dihydroxy-1-phenylsulfonyl-1,5-diazacyclooctane (2.92 g, 7.76 mmol; see Example 2 above). The reaction mixture was heated for 24 hours at 95° C. The temperature was adjusted to 60° C., and toluene (40 mL) was added. Sodium hydroxide solution was then added (150 mL of 5 M), causing the internal temperature to rise to 85° C. The pH of the reaction mixture was then checked, and was found to be 2. A few pellets of solid sodium hydroxide were then added. The pH was measured again, and was found to be 13. The layers were separated, and the aqueous phase extracted with toluene (50 mL). The organic phases were combined, dried (MgSO4), filtered and concentrated in vacuo to give the title compound as an orange-brown oil.

¹H NMR (300 MHz, DMSO-d₆) δ 7.25 (m, 5H), 4.38 (s, 1H), 3.69 (s, 1H), 3.49 (m, 2H), 3.34 (m, 2H), 2.99 (d, J=13.8 Hz, 1H), 2.86 (m, 2H), 2.74 (m, 1H), 2.64 (m, 2H).

### Abbreviations

Et=ethyl
eq.=equivalents
h=hour(s)
IMS=industrial methylated spirit (denatured ethanol)
IPA=iso-propyl alcohol
kPa=kiloPascal
Me=methyl
MIBC=methyl-2-pentanol
min.=minute(s)
Pd/C=palladium on carbon
Pt/C=platinum on carbon

Prefixes n-, s-, t- and tert- have their usual meanings: normal, secondary, iso, and tertiary.

1. A process for the preparation of a compound of formula I, 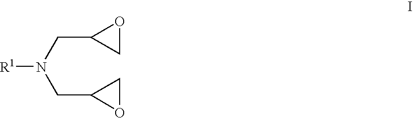
or a salt and/or solvate thereof,
wherein R¹ represents an amino protective group or a structural fragment of formula Ia, 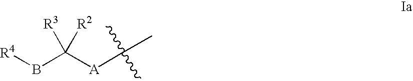
in which
R² represents H, halo, C₁₋₆ alkyl, —OR⁵, -E-N(R⁶)R⁷ or, together with R³, represents ═O;
R³ represents H, C₁₋₆ alkyl or, together with R², represents ═O;
R⁵ represents H, C₁₋₆ alkyl, -E-aryl, -E-Het¹, —C(O)R^{8a}, —C(O)OR^{8b} or —C(O)N(R^{9a})R^{9b};
R⁶ represents H, C₁₋₆ alkyl, -E-aryl, -E-Het¹, —C(O)R^{8a}, —C(O)OR^{8b}, —S(O)₂R^{8c}, —[C(O)]ₚN(R^{9a})R^{9b} or —C(NH)NH₂;
R⁷ represents H, C₁₋₆ alkyl, -E-aryl or —C(O)R^{8d};
R^{8a} to R^{8d} independently represent, at each occurrence when used herein, C₁₋₆ alkyl (optionally substituted by one or more substituents selected from halo, aryl and Het²), aryl, Het³, or R^{8a} and R^{8d} independently represent H;
R^{9a} and R^{9b} independently represent, at each occurrence when used herein, H or C₁₋₆ alkyl (optionally substituted by one or more substituents selected from halo, aryl and Het⁴), aryl, Het⁵, or together represent C₃₋₆ alkylene, optionally interrupted by an O atom;
E represents, at each occurrence when used herein, a direct bond or C₁₋₄ alkylene;
p represents 1 or 2;
A represents a direct bond, -J-, -J-N(R^{10a})—, -J-S(O)₂N(R^{10b})—, -J-N(R^{10c})S(O)₂— or -J-O— (in which latter four groups -J is attached to the oxabispidine nitrogen);
B represents —Z—{[C(O)]ₐC(H)(R^{11a})}_{b}—, —Z—[C(O)]_{c}N(R^{11b})—, —Z—N(R^{11c})S(O)₂—, —Z—S(O)₂N(R^{11d})—, —Z—S(O)ₙ—, —Z—O— (in which latter six groups, Z is attached to the carbon atom bearing R² and R³), —N(R^{11e})—Z—, —N(R^{11f})S(O)₂—Z—, —S(O)₂N(R^{11g})—Z— or —N(R^{11h})C(O)O—Z— (in which latter four groups, Z is attached to the R⁴ group);
J represents C₁₋₆ alkylene optionally interrupted by —S(O)₂N(R^{10d})— or —N(R^{10c})S(O)₂— and/or optionally substituted by one or more substituents selected from —OH, halo and amino;
Z represents a direct bond or C₁₋₄ alkylene, optionally interrupted by —N(R¹¹ⁱ)S(O)₂— or —S(O)₂N(R^{11j})—;
a, b and c independently represent 0 or 1;
n represents 0, 1 or 2;
R^{10a} to R^{10e} independently represent, at each occurrence when used herein, H or C₁₋₆ alkyl;
R^{11a} represents H or, together with a single ortho-substituent on the R⁴ group (ortho-relative to the position at which the B group is attached), R^{11a} represents C₂₋₄ alkylene optionally interrupted or terminated by O, S, N(H) or N(C₁₋₆ alkyl);
R^{11b} represents H, C₁₋₆ alkyl or, together with a single ortho-substituent on the R⁴ group (ortho-relative to the position at which the B group is attached), R^{11b} represents C₂₋₄ alkylene;
R^{11c} to R^{11j} independently represent, at each occurrence when used herein, H or C₁₋₆ alkyl;
R⁴ represents phenyl or pyridyl, both of which groups are optionally substituted by one or more substituents selected from —OH, cyano, halo, nitro, C₁₋₆ alkyl (optionally terminated by —N(H)C(O)OR^{12a}), C₁₋₆ alkoxy, —N(R^{13a})R^{13b}, —C(O)R^{13c}, —C(O)OR^{13d}, —C(O)N(R^{13e})R^{13f}, —N(R^{13g})C(O)R^{13h}, —N(R¹³ⁱ)C(O)N(R^{13j})R^{13k}, —N(R^{13m})S(O)₂R^{12b}, —S(O)₂N(R¹³ⁿ)R^{13o}, —S(O)₂R^{12c}, —OS(O)₂R^{12d} and/or aryl;
and an ortho-substituent (ortho-relative to the attachment of B) may
(i) together with R^{11a}, represent C₂₋₄ alkylene optionally interrupted or terminated by O, S, N(H) or N(C₁₋₆ alkyl), or
(ii) together with R^{11b}, represent C₂₋₄ alkylene;
R^{12a} to R^{12d} independently represent C₁₋₆ alkyl;
R^{13a} and R^{13b} independently represent H, C₁₋₆ alkyl or together represent C₃₋₆ alkylene, resulting in a four- to seven-membered nitrogen-containing ring;
R^{13c} to R^{13o} independently represent H or C₁₋₆ alkyl; and
Het¹ to Het⁵ independently represent, at each occurrence when used herein, five- to twelve-membered heterocyclic groups containing one or more heteroatoms selected from oxygen, nitrogen and/or sulfur, which heterocyclic groups are optionally substituted by one or more substituents selected from ═O; —OH, cyano, halo, nitro, C₁₋₆ alkyl, C₁₋₆ alkoxy, aryl, aryloxy, —N(R^{14a})R^{14b}, —C(O)R^{14c}, —C(O)OR^{14d}, —C(O)N(R^{14e})R^{14f}, —N(R^{14g})C(O)R^{14h}, —S(O)₂N(R¹⁴ⁱ)(R^{14j}) and/or —N(R^{14k})S(O)₂R^{14l};
R^{14a} to R^{14l} independently represent C₁₋₆ alkyl, aryl or R^{14a} to R^{14k} independently represent H;
provided that:
(a) when R³ represents H or C₁₋₆ alkyl; and
A represents -J-N(R^{10a})— or -J-O—, then:
(i) J does not represent C₁ alkylene or 1,1-C₂₋₆ alkylene; and
(ii) B does not represent —N(R^{11b})—, —N(R^{11c})S(O)₂—, —S(O)ₙ—, —O—, —N(R^{11e})—Z—, —N(R^{11f})S(O)₂—Z— or —N(R^{11h})C(O)O—Z—; and
(b) when R² represents —OR⁵ or -E-N(R⁶)R⁷ in which E represents a direct bond, then:
(i) A does not represent a direct bond, -J-N(R^{10a}) -J-S(O)₂N(R^{12h})— or -J-O—; and
(ii) B does not represent —N(R^{11b})—, —N(R^{11c})S(O)₂—, —S(O)ₙ—, —O—, —N(R^{11e})—Z, —N(R^{11f})S(O)₂—Z— or —N(R^{11h})C(O)O—Z—; and
(c) when A represents -J-N(R^{10c})S(O)₂, then J does not represent C₁ alkylene or 1,1-C₂₋₆ alkylene; and
(d) when R³ represents H or C₁₋₆ alkyl and A represents -J-S(O)₂N(R^{10b}), then B does not represent —N(R^{11b})—, —N(R^{11c})S(O)₂—, —S(O)ₙ—, —O—, —N(R^{11e})—Z—, —N(R^{11f})S(O)₂—Z— or —N(R^{11h})C(O)O—Z—; and
wherein each aryl and aryloxy group, unless otherwise specified, is optionally substituted;
which process comprises reaction in the presence of an aqueous solvent system of: one equivalent of a compound of formula II,
H₂N—R¹ II
or a salt and/or solvate thereof, wherein R¹ is as hereinbefore defined,
with at least two equivalents of a compound of formula III, 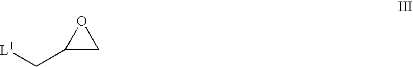
wherein L¹ represents a leaving group,
and at least two equivalents of base,
wherein the reaction is performed by addition of base to an aqueous mixture of the compounds of formulae II and III, the period of base addition comprising:
(a) a first period, during which the pH of the reaction mixture is raised to between pH 10 and pH 13; and then
(b) a second period, during which the pH of the reaction mixture is controlled such that it is maintained between pH 10 and pH 13,
wherein the time ratio of the first to second period is 1:5 or less.

2. A process as claimed in claim 1, wherein R¹ represents an amino protective group.

3. A process as claimed in claim 2, wherein R¹ represents a benzenesulfonyl group.

4. A process as claimed in any one of the preceding claims wherein the aqueous solvent system is water.

5. A process for the preparation of a compound of formula IV, 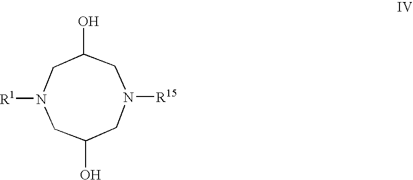
or a salt and/or solvate thereof;
wherein R¹⁵ represents H, an amino protective group or a structural fragment of formula Ia, as defined in claim 1; and
R¹ is as defined in claim 1;
which process comprises reaction of a compound of formula I, as defined in claim 1, with a compound of formula V,
H₂N—R¹⁵ V
or a salt and/or solvate thereof,
wherein R¹⁵ is as defined above; and
wherein the compounds of formula I and V are added, separately, simultaneously and at a substantially equivalent rate of moles per minute, to a reaction vessel containing solvent.

6. A process for the preparation of a compound of compound of formula IV, as defined in claim 5, or a salt and/or solvate thereof, which process comprises:
(i) a process as defined in any one of claims 1 to 4 for the preparation of a compound of formula I, as defined in claim 1; and then
(ii) reaction of the resulting compound of formula I, or a salt and/or solvate thereof, with a compound of formula V, as defined in claim 5, wherein the compounds of formulae I and V are added, separately, simultaneously and at a substantially equivalent rate of moles per minute, to a reaction vessel containing solvent.

7. A process as claimed in claim 5 or claim 6, wherein R¹⁵ represents an amino protective group.

8. A process as claimed in claim 7, wherein R¹⁵ represents benzyl.

9. A process as claimed in any one of claims 5 to 8, wherein the solvent is a water-miscible organic solvent.

10. A process as claimed in claim 9, wherein the solvent is IMS or methanol.

11. A process as claimed in any one of claims 5 to 9, wherein the simultaneous addition of compounds of formulae I and V to a reaction vessel containing solvent is effected in either a portion-wise or continuous manner.

12. A process for the preparation of a compound of formula VI, 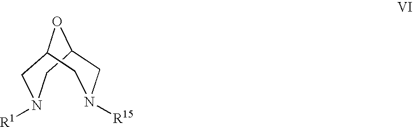
or a salt and/or solvate thereof;
wherein R¹ is as defined in claim 1 and R¹⁵ is as defined in claim 5;
which process comprises a process as described in any one of claims 5 to 10 for the preparation of a compound of formula IV, followed by dehydrative cyclisation of that compound.

13. A process for the preparation of a compound of formula VII, 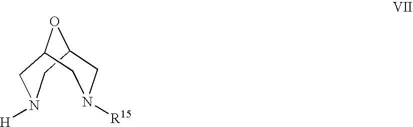
or a salt and/or solvate thereof, wherein R¹⁵ is as defined in claim 5, which process comprises sulfuric acid-catalysed hydrolysis of a compound of formula VIa 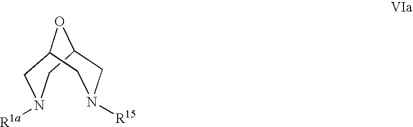
or a salt and/or solvate thereof, wherein R^{1a} represents benzenesulfonyl optionally substituted on the benzene ring by one or more substituents selected from C₁₋₄ alkyl, C₁₋₄ alkoxy and halo, and R¹⁵ is as defined in claim 5.

14. A process for the preparation of a compound of formula VII, as defined in claim 13, which process comprises
(a) a process as described in claim 12 for the preparation of a compound of formula VI in which R¹ represents R^{1a}, wherein R^{1a} is as defined in claim 13, followed by
(b) reaction of the resulting compound of formula VI, or a salt and/or solvate thereof, with concentrated sulfuric acid.

15. A process for the preparation of, a compound of formula VII, or a salt and/or solvate thereof, as defined in claim 13, which process comprises reaction of a compound of formula IVa, 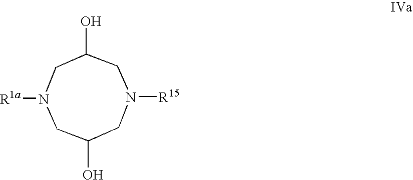
or a salt and/or solvate thereof, wherein R^{1a} is as defined in claim 13 and R¹⁵ is as defined in claim 5, with concentrated sulfuric acid.

16. A process for the preparation of a compound of formula VII, or a salt and/or solvate thereof, as defined in claim 13 which process comprises:
(a) a process as described in any one of claims 5 to 11 for the preparation of a compound of formula IV in which R¹ represents R^{1a}, wherein R^{1a} is as defined in claim 13, followed by
(b) reaction of the resulting compound of formula IV, or a salt and/or solvate thereof, with concentrated sulfuric acid.

17. A process as claimed in any one of claims 13 or claim 16, wherein the sulfuric acid has a H₂SO₄ content of more than 40% by weight.

18. A process as claimed in any one of claims 1 and 4 to 12, wherein R¹ represents 2-phenethyl (optionally substituted in the phenyl part by one or more substituents selected from halo and C₁₋₄ alkoxy).

19. A process as claimed in any one of claims 5, 6 and 9 to 12, wherein R¹ represents benzyl.

20. A process as claimed in any one of claims 5, 6, 9 to 17 and 19, wherein R¹⁵ represents 2-phenethyl (optionally substituted in the phenyl part by one or more substituents selected from halo and C₁₋₄ alkoxy).
